(19) United States
(12) Patent Application Publication
(10) Pub. No.: US 2025/0049982 A1
(43) Pub. Date: Feb. 13, 2025

(54) COMPOSITION COMPRISING A BIOMATERIAL-BASED POROUS MATERIAL COATED WITH A POWDER

(71) Applicant: MEDSKIN SOLUTIONS DR. SUWELACK AG, Billerbeck (DE)

(72) Inventors: Marco WOGRAM, Oststeinbek (DE); Annalena VÖLKER, Nottuln (DE)

(73) Assignee: MEDSKIN SOLUTIONS DR. SUWELACK AG, Billerbeck (DE)

(21) Appl. No.: 18/719,921

(22) PCT Filed: Dec. 19, 2022
(86) PCT No.: PCT/EP2022/086692

§ 371 Date: Jun. 14, 2024

(30) Foreign Application Priority Data
Dec. 17, 2021 (EP) 21215766.3
Dec. 17, 2021 (EP) 21215771.3

(51) Int. Cl. A61L 15/42; A61L 15/18; A61L 15/32

(52) U.S. Cl. CPC A61L 15/425; A61L 15/18; A61L 15/325; A61L 2400/04; A61L 2420/02

(57) ABSTRACT

The present invention relates to a composition comprising a biomaterial-based porous material coated with a powder and a method of making such compositions The invention further relates to a method of controlling bleeding and/or leakage of other body fluids in surgical procedures or treatment of an injury selected from the group consisting of a wound, a hemorrhage, damaged tissue and/or bleeding tissue as well as skin treatment comprising administering such compositions.

### TECHNICAL FIELD

The present invention relates to a composition comprising a biomaterial-based porous material coated with a powder and a method of making such compositions. The invention further relates to a method of controlling bleeding and/or leakage of other body fluids in surgical procedures or treatment of an injury selected from the group consisting of a wound, a hemorrhage, damaged tissue and/or bleeding tissue as well as skin treatment comprising administering such compositions.

### BACKGROUND OF THE INVENTION

A variety of coating techniques are employed in order to deposit a material onto a substrate, which include chemical or physical vapor deposition, electrochemical techniques, spraying, slot-die coating, etc. In particular, the use of electrostatic powder coating techniques to coat electrically conductive substrates, such as metals, is well known. By this method, a powder coating material is statically charged and then sprayed or blown onto a surface of a conductive material to which it adheres. The material is impregnated with the powder by means of electrostatic attraction between the positively charged or ionized powder and negatively charged surface of the conductive material or vise versa. This method is particularly used for painting metal articles.

However, in recent years, there has been an increase of the use of polymeric materials in the manufacture of articles, particularly in applications requiring reductions in weight and improved corrosion resistance. At the same time, typically such polymers have a poor conductivity to be efficiently coated by the above method, since they cannot be efficiently electrostatically charged to attract charged powder particles.

One way to improve the conductivity of polymers is employing conductive primer compositions to the polymers. WO 2004/069942 provides an example of such primer compositions. However, depending on the particular primer employed, the tuned polymer may have less favorable physical and chemical properties, e.g. surface smoothness, physicochemical stability, etc., making the tuned material less suitable for a particular application. Additionally, such primers compositions may contain volatile organic solvents, the emission of which during the priming process may be undesirable, as well as environmentally unfriendly.

A different approach is based on subjecting the poorly conductive article and a coating powder to an electric field, generated by an external source. In the international application WO 99/22920 a method of impregnating a fibrous or filamentary network with powder is described, in particular, for producing a composite material. In this process, the powder and the network of fibers or filaments are subjected to an alternating electric field, which two electrodes connected to the same voltage generator produce between them. Each electrode has the shape of a metal plate. In application EP 1526214 the electric field is created by a plurality of electrodic tubes. Further arrangements of electrodes are described in WO 2007/110524 and EP 2231209 B1.

An advantage of applying of electrostatic powder coating to a porous biomaterial, such as collagen, lies in that the density of the biomaterial matrix does not change. As a result, the capabilities of the biomaterial matrix to uptake water and/or to release the drug substance remain intact. In contrast, applying solution coating techniques, such as slot-die coating, to a porous biomaterial matrix results in an essential increase of the density of the material.

Collagen-based pads, tissues or sponges have been particularly used for many years to improve wound healing or to stop bleeding (see U.S. Pat. No. 4,600,574 A, WO 2004/028404, U.S. Pat. No. 5,614,587 A, EP 2939697 B1). Their mechanism of action in hemostasis is based on platelets aggregation and activation, the formation of thrombin on the surface of activated platelets and the formation of a hemostatic fibrin clot by the catalytic action of thrombin on fibrinogen.

Certain functionalities of polymers, e.g. diffusion properties, water uptake, conductivity are oftenly pH-sensitive. For example, presence of functional groups in a polymer, such as OH, COOH or NH₂ may impact water diffusivity in the polymeric films, which in turn may result in a clear pH-dependence of the drug release kinetics from coated pellets. Therefore, tuning pH properties of a polymeric substrate is often necessary for improvement of efficacy of an active substance released from a coated dosage form.

### DESCRIPTION OF THE INVENTION

In one aspect, the present invention relates to a composition comprising a porous material, wherein said porous material comprises a biomaterial and comprises a plurality of open and interconnected pores with pore surfaces,
wherein said porous material has a density in the range of from 0.01 to 1 g/cm³,
wherein said pores have an average diameter in the range of from 15 to 70 μm, characterized in that said porous material is coated with an electrostatically chargeable powder comprising particles,
wherein said particles have an average size in the range of from 50 to 100 μm, and wherein the total quantity of the coating is in the range of from 2 to 100 g/m².

In one embodiment, the composition according to the present invention comprises a porous material, wherein said porous material comprises collagen and comprises a plurality of open and interconnected pores with pore surfaces,
wherein said porous material has a density in the range of from 0.01 to 1 g/cm³,
wherein said pores have an average diameter in the range of from 15 to 70 μm, characterized in that said porous material is coated with an electrostatically chargeable powder comprising particles, wherein said powder contains at least 95% by weight of sodium bicarbonate (NaHCO₃),
wherein said particles have an average size in the range of from 50 to 100 μm,
wherein the total quantity of the coating is in the range of from 2 to 100 g/m² of the outer surface of said porous material, and
wherein the coating adjusts the pH at the surface of the composition to a range of from 3,0 to 9,0, preferably in the range of from 6,0 to 8,0, more preferably in the range of from 6,5 to 7,5.

In the composition according to the present invention the surface of the porous material is covered with an essentially even layer of powder particles, wherein the powder predominantly remains on the surface of the porous material, with only a minor part of the particles going inside the pores. This is achieved due to size classification of the powder particles, which have an average size predominantly higher than an average diameter of a pore size. As a result, the pores of the biomaterial remain unblocked and the coated tuned biomaterial retains its capabilities to uptake water and/or to release the drug substance remain intact. This is in particular illustrated by Example 2. As can be seen from Example 2, the essential technical parameters of the coated material such as residual moisture, tensile strength and water absorption remain essentially intact compared to the uncoated collagen.

The powder coating does two things in one. It allows to functionalize the surface with specific properties like pH-adjustment, adhesive application, whatever actually comes in a powder but at the same time the adherence of the powder through electrostatics and the choice of particle size distribution allows for keeping the original properties like tensiles strength, pore size and pore openness (hence the stable water absorption compared to uncoated material), wicking behavior etc.

In another aspect, the present invention relates to a method of making the composition comprising a porous material, comprising the steps of:
a) Providing a porous material wherein said porous material comprises a biomaterial and comprises a plurality of open and interconnected pores with pore surfaces, wherein said porous material has a density in the range of from 0.01 to 1 g/cm³, wherein said pores have an average diameter in the range of from 15 to 70 μm;
b) Proving an electrostatically chargeable powder comprising particles, wherein said particles have an average size in the range of from 50 to 100 μm;
c) Positioning said porous material and said powder between opposite-facing electrodes in a coating device, wherein said coating device is able to generate an electric field through the porous medium, and wherein said device has an area for storing said powder;
d) Electrostatic depositing of said powder on the surface of said porous material by means of subjecting said powder and said porous material to an electric field produced by the opposing electrodes, wherein said electric field moves said particles towards said porous material.

In one embodiment, a method of making the composition comprising a porous material according to the present invention comprises the steps of:
a) Providing a porous material wherein said porous material comprises collagen and comprises a plurality of open and interconnected pores with pore surfaces, wherein said porous material has a density in the range of from 0.01 to 1 g/cm³, wherein said pores have an average diameter in the range of from 15 to 70 μm;
b) Proving an electrostatically chargeable powder comprising particles, wherein said powder contains at least 95% by weight of sodium bicarbonate (NaHCO₃) and wherein said particles have an average size in the range of from 50 to 100 μm;
c) Positioning said porous material and said powder between opposite-facing electrodes in a coating device, wherein said coating device is able to generate an electric field through the porous medium, and wherein said device has an area for storing said powder;
d) Electrostatic depositing of said powder on the surface of said porous material by means of subjecting said powder and said porous material to an electric field produced by the opposing electrodes, wherein said electric field moves said particles towards said porous material.

### DETAILED DESCRIPTION

In one aspect, the present invention relates to a composition comprising a porous material, wherein said porous material comprises biomaterial and comprises a plurality of open and interconnected pores with pore surfaces,
wherein said porous material has a density in the range of from 0.01 to 1 g/cm³,
wherein said pores have an average diameter in the range of from 15 to 70 μm, characterized in that said porous material is coated with an electrostatically chargeable powder comprising particles,
wherein said particles have an average size in the range of from 50 to 100 μm, and
wherein the total quantity of the coating is in the range of from 2 to 100 g/m².

In one embodiment, the composition according to the present invention comprises a porous material, wherein said porous material comprises biomaterial and comprises a plurality of open and interconnected pores with pore surfaces,
· wherein said porous material has a density in the range of from 0.01 to 1 g/cm³,
· wherein said pores have an average diameter in the range of from 15 to 70 μm, characterized in that said porous material is coated with an electrostatically chargeable powder comprising particles,
· wherein said particles have an average size in the range of from 50 to 100 μm, and
· wherein the total quantity of the coating is in the range of from 2 to 100 g/m².

In one embodiment, the porous material comprises at least 90% by weight biomaterial, preferably the porous material comprises at least 95% by weight biomaterial, more preferably the porous material comprises at least 96% by weight biomaterial, more preferably the porous material comprises at least 97% by weight biomaterial, more preferably the porous material comprises at least 98% by weight biomaterial, most preferred the porous material comprises at least 99% by weight biomaterial. In one embodiment the biomaterial is collagen.

In one embodiment the density of the porous material is in the range of from 0.01 to 1 g/cm³, preferably of from 0,02 to 0.05 g/cm³, more preferably in the range of from 0,02 to 0,04 g/cm³, most preferred in the range of from 0,022 to 0,03 g/cm³.

In one embodiment, the pores in the porous material have an average diameter in the range of from 15 to 70 μm, preferably in the range of from 25 to 65 μm.

In one embodiment the porous material has a density in the range of from 0.01 to 1 g/cm³, wherein the pores have an average diameter in the range of from 15 to 70 μm. In a preferred embodiment the porous material has a density in the range of from 0,02 to 0.05 g/cm³, wherein the pores have an average diameter in the range of from 25 to 65 μm. In a more preferred embodiment the porous material has a density in the range of from 0,02 to 0,04 g/cm³, wherein the pores have an average diameter in the range of from 25 to 65 μm.

In one embodiment said composition comprising a porous material is characterized by water absorption of 20 to 40 g per g of porous material. In one embodiment said composition comprising a porous material is characterized by water absorption which is at least 80%, preferably at least 90%, more preferably at least 95%, even more preferably at least 98%, even more preferably at least 99% in relation to water absorption of the uncoated porous material.

In one embodiment the particles of the electrostatically chargeable powder have an average size in the range of from 50 to 100 μm, preferably in the range of from 55 to 85 μm.

In one embodiment the total quantity of the coating in the composition is in the range of from 2 to 100 g/m², preferably in the range of from 2 to 20 g/m², more preferably in the range of from 3,5 to 9 g/m².

In one embodiment the total quantity of the coating in the composition is in the range of from 10 to 100 g/m².

In one embodiment the total quantity of the coating in the composition is in the range of from 2 to 100 g/m² of the outer surface of said porous material, preferably in the range of from 2 to 20 g/m² of the outer surface of said porous material, more preferably in the range of from 3,5 to 9 g/m² of the outer surface of said porous material.

In one embodiment the total quantity of the coating in the composition is in the range of from 10 to 100 g/m² of the outer surface of said porous material.

In one embodiment, the average size of at least 70% of the particles of the electrostatically chargeable powder exceeds the average diameter of the pores in the porous material of the composition. In one embodiment, the average size of at least 80% of the particles of the electrostatically chargeable powder exceeds the average diameter of the pores in the porous material of the composition. In one embodiment, the average size of at least 90% of the particles of the electrostatically chargeable powder exceeds the average diameter of the pores in the porous material of the composition. So, the particles of the powder predominantly remain on the surface of the porous material, wherein only a minor part of the particles penetrates the porous material via the pores.

In one embodiment, the coating in the composition adjusts the pH at the surface of the composition to a range of from 3,0 to 9,0, preferably to a range of from 6,0 to 8,0, more preferably to a range of from 6,5 to 7,5.

In one embodiment, the coating in the composition adjusts the pH at the surface of the composition to a range of from 3,5 to 4,5.

The pH can be adjusted to a suitable range by means of selection of suitable powder source. For example, sodium bicarbonate has been found particularly suitable for the adjustment the pH at the surface of the composition to a range of from 3,0 to 9,0, in particular to a range of from 6,0 to 8,0.

The pH on the surface of the material can be measured by any suitable surface pH electrode. In particular, the pH on the surface of the composition can be measured by a pH electrode with a flat membrane and polymer electrolyte, e.g. WTW SenTix® Sur, which is suitable for measurements on smooth surfaces.

In one embodiment, the porous material is a biomaterial.

In one embodiment, the porous material is selected from the group comprising natural and/or synthetic polymers or mixtures thereof, in particular polysaccharides, glucosaminoglycanes, proteins or mixtures thereof.

In one embodiment, the porous material is selected from the group consisting of collagen, alginate, for example, calcium alginate, or a mixture thereof.

In one embodiment, the porous material is collagen.

In one embodiment, the porous material is alginate, in particular calcium alginate.

In one embodiment the porous material is a mixture of collagen and calcium alginate. In one embodiment, the porous material comprises collagen by weight in the range of from 80 to 98% and calcium alginate by weight in the range of from 2 to 20%. In a preferred embodiment, the porous material comprises collagen by weight in the range of from 85 to 95% and calcium alginate by weight in the range of from 5 to 15%. In particular, the porous material comprises collagen about 90% by weight and calcium alginate about 10% by weight.

In one embodiment, the collagen in the porous material of the composition is animal derived native collagen with a triple helical structure.

In one embodiment, the collagen in the porous material of the composition is selected from the group comprising type 1 collagen, type 3 collagen, type 5 collagen or a mixture thereof.

In one embodiment, the composition of the present invention is in the form of a sheet or a 3D form. In one embodiment, the composition of the present invention is in the form of a sheet. In one embodiment, the composition of the present invention is in the form of a 3D form.

In one embodiment, the porous material is essentially flat, i.e. the thickness of the porous material throughout the length and the width of the material does not deviate from the average thickness of the porous material by more than +20%, preferably +10%. For example, with an average thickness of 1 mm the thickness of the essentially flat material throughout its length and width remains in the range of from 0,8 to 1.2 mm, preferably in the range of from 0,9 to 1.1 mm. With an average thickness of 2 mm the thickness of the essentially flat material throughout its length and width remains in the range of from 1,6 to 2.4 mm, preferably in the range of from 1,8 to 2.2 mm.

In one embodiment, the electrostatically chargeable powder comprises a compound selected from the group consisting of a salt, a glucose polysaccharide, glucose, a modified glucose, an enzyme, a collagen, hyaluronic acid, a metal or a metal oxide.

In one embodiment, the electrostatically chargeable powder comprises a salt selected from the group comprising sodium bicarbonate (NaHCO₃), magnesium carbonate (MgCO₃), calcium carbonate (CaCO₃), sodium lactate, sodium citrate and sodium iodide (Nal), or a mixture thereof. In one embodiment, the electrostatically chargeable powder comprises a salt selected from the group comprising magnesium carbonate (MgCO₃), calcium carbonate (CaCO₃), sodium lactate, sodium citrate, and sodium iodide (Nal) or a mixture thereof.

In one embodiment, the electrostatically chargeable powder comprises a salt, which is not sodium bicarbonate (NaHCO₃).

In one embodiment the salt has a monovalent cation and a monovalent anion, for example, sodium bicarbonate (NaHCO₃). In one embodiment, the salt has a divalent cation and a monovalent anion, for example calcium carbonate (CaCO₃).

In one embodiment the salt is sodium bicarbonate (NaHCO₃). Preferably, the salt comprises sodium bicarbonate (NaHCO₃) at least 95% by weight, more preferably at least 96% by weight, even more preferably at least 97% by weight, even more preferably at least 98% by weight, even more preferably at least 99% by weight, in particular at least 99,5% by weight, with respect to the dry compound. The salt comprising sodium bicarbonate may contain minor amounts of sodium iodide (Nal) and/or magnesium carbonate (MgCO₃). In one embodiment, the salt comprising sodium bicarbonate contains up to 5% by weight sodium iodide (Nal) with respect to the dry compound. In one embodiment, the salt comprising sodium bicarbonate contains up to 5% by weight magnesium carbonate (MgCO₃) with respect to the dry compound. In one embodiment the salt consists of sodium bicarbonate (NaHCO₃).

In one embodiment sodium bicarbonate comprises less than 40% moisture by weight, preferably less 35% moisture by weight, more preferably less than 30% moisture by weight.

In one embodiment the electrostatically chargeable powder comprises the glucose polysaccharide is selected from the group comprising cellulose and starch.

In one embodiment the electrostatically chargeable powder comprises a modified glucose. In one embodiment the modified glucose is glucose with the radionuclide fluorine-18 (18F) in place of the hydroxyl group on the 2 carbon (FDG).

In one embodiment the electrostatically chargeable powder comprises an enzyme.

In one embodiment the electrostatically chargeable powder comprises collagen. In one the collagen in the porous material of the composition is selected from the group comprising type 1 collagen, type 3 collagen, type 5 collagen or a mixture thereof.

In one embodiment the electrostatically chargeable powder comprises hyaluronic acid.

In one embodiment the electrostatically chargeable powder comprises a metal, such as titanium.

In one embodiment the electrostatically chargeable powder comprises the metal oxide, for example, titanium dioxide (TiO₂).

In one embodiment the thickness of the porous material in the composition is in the range of from 0.5 to 10 mm, preferably in the range of from 1 to 5 mm.

In one embodiment, the composition is in a form of a sheet or a 3D form, wherein one side of the sheet or the 3D form is coated by the powder coating. In one embodiment, the composition is in a form of a sheet, wherein two sides of the sheet are coated by the powder coating.

The composition of the present invention may be further coated by an additional layer of a polymer or wax. Non-limiting examples of polymers include polyurethane and polyalkylene oxide polymers. In one embodiment the polymer is a polyalkylene oxide polymer, preferably a PEG comprising polymer, e.g.

a multi-electrophilic polyalkylene oxide polymer, e.g. a multi-electrophilic PEG, such as pentaerythritolpoly(ethyleneglycol) ether tetrasuccinimidyl glutarate (COH 102). In one embodiment, the composition is in the form of a dressing. In one embodiment, the composition according to any of the preceding embodiments, which is further coated with an additional layer of a polymer is suitable for use in controlling bleeding and/or leakage of other body fluids in surgical procedures or for treatment of an injury selected from the group consisting of a wound, a hemorrhage, damaged tissue and/or bleeding tissue. In one embodiment, the additional layer of a polymer or wax in the composition of the present invention, such as polyalkylene oxide polymer, is on top of the powder coating.

In another aspect, the present invention relates to a method of making the composition comprising a porous material, comprising the steps of:
a) Providing a porous material wherein said porous material comprises a biomaterial and comprises a plurality of open and interconnected pores with pore surfaces, wherein said porous material has a density in the range of from 0.01 to 1 g/cm³, wherein said pores have an average diameter in the range of from 15 to 70 μm;
b) Proving an electrostatically chargeable powder comprising particles, wherein said particles have an average size in the range of from 50 to 100 μm;
c) Positioning said porous material and said powder between opposite-facing electrodes in a coating device, wherein said coating device is able to generate an electric field through the porous medium, and wherein said device has an area for storing said powder;
d) Electrostatic depositing of said powder on the surface of said porous material by means of subjecting said powder and said porous material to an electric field produced by the opposing electrodes, wherein said electric field moves said particles towards said porous material.

In another embodiment, the method of making the composition comprising a porous material, comprises the steps of:
a) Providing a porous material wherein said porous material comprises collagen and comprises a plurality of open and interconnected pores with pore surfaces, wherein said porous material has a density in the range of from 0.01 to 1 g/cm³, wherein said pores have an average diameter in the range of from 15 to 70 μm;
b) Proving an electrostatically chargeable powder comprising particles, wherein said powder contains at least 95% by weight of sodium bicarbonate (NaHCO₃) and wherein said particles have an average size in the range of from 50 to 100 μm;
c) Positioning said porous material and said powder between opposite-facing electrodes in a coating device, wherein said coating device is able to generate an electric field through the porous medium, and wherein said device has an area for storing said powder;
d) Electrostatic depositing of said powder on the surface of said porous material by means of subjecting said powder and said porous material to an electric field produced by the opposing electrodes, wherein said electric field moves said particles towards said porous material.

In one embodiment, the powder in step d) in the method of making the composition comprising a porous material is in a fluidized state by means of providing a stream of a fluidizing gas into the area for storing the powder in the coating device. Fluidizing helps the particles to get separated from each other and to make the charging and discharging onto the flat membrane easier. In particular, the fluidizing gas can be air, nitrogen, a noble gas, such as helium, neon, argon, krypton or xenon, or mixtures thereof. In one embodiment the fluidizing gas is air. In one embodiment the fluidizing gas is nitrogen. In one embodiment the fluidizing gas is argon.

In one embodiment, the voltage applied to the opposing-facing electrodes in the method of making the composition comprising a porous material is in the range of from 20 to 250 kV. In a preferred embodiment, the voltage applied to the opposing-facing electrodes in the method of making the composition comprising a porous material is in the range of from 30 to 60 kV. In a more preferred embodiment, the voltage applied to the opposing-facing electrodes in the method of making the composition comprising a porous material is in the range of from 45 to 55 kV. Most preferred, the voltage applied to the opposing-facing electrodes in the method of making the composition comprising a porous material is about 50 kV.

In one embodiment, in the method of making the composition comprising a porous material the opposing-facing electrodes in the coating device are arranged in the way that at least first electrode is positioned in a proximity to the porous material, and at least second electrode is positioned to be in direct or indirect contact (e.g. separated by a polymer membrane) with the area for storing powder in the coating device. In one embodiment, the at least first electrode is a cathode and the at least second electrode is anode. In one embodiment, the at least first electrode is a anode and the at least second electrode is cathode.

In one embodiment the at least first electrode is positioned behind the porous material relative to the area for storing powder, which, in turn, is positioned behind the at least second electrode. In one embodiment the at least first electrode is positioned above the porous material relative to the area for storing powder, which, in turn, is positioned above the at least second electrode. In one embodiment, the at least first electrode and the at least second electrode are positioned essentially parallel to each other.

In one embodiment, the at least first electrode is a metallic plate.

In one embodiment, the at least second electrode is a multiplicity of electrodic wires. The number of electrodes needed, their size, spacing and further arrangements in the coating device are determined by a number of parameters. These parameters include the diameter, electrical conductivity, type of powder, and the voltage applied.

In one embodiment, the particles of the powder are charged negatively at the at least second electrode, which is cathode, and are electrostatically attracted by the at least first electrode, which is anode. In one embodiment, the particles of the powder are charged positively at the at least second electrode, which is anode, and are electrostatically attracted by the at least first electrode, which is cathode.

In one embodiment, the coating device is connected to a voltage generator.

In one embodiment, in the method of making the composition comprising a porous material the coating device comprises a means for moving the porous material horizontally across the powder to at least partly displace a portion of the powder across the porous material. With this embodiment a continuous operating mode, in which the powder is evenly distributed across the porous material thus providing a coating of uniform thickness, is assured. In one embodiment the moving means is at least one roll, e.g. a rolling drum, wherein the porous material is wound cylindrically about a center axis with the possibility of removal of the material from the center or inner periphery of the roll. In one embodiment, the dwell time over the fluid bed is in the range of from 1 to 10 seconds, preferably in the range of from 2 to 9 seconds, more preferably in the range of from 3 to 6 seconds.

In one embodiment, the volumetric flow rate of the fluidizing gas in the method of making the composition comprising a porous material is in the range of from 40 to 120 l/min, preferably in the range of from 60 to 100 l/min, in particular about 80 l/min.

In one embodiment, the density of the powder in the method of making the composition comprising a porous material is in the range of from 2,0 to 2,5 g/cm³, preferably in the range of from 2,1 to 2,3 g/cm³, in particular about 2,2 g/cm³.

In one embodiment, the distance between said area for storing the powder and the porous material in the coating device is in the range of from 80 to 200 mm, preferably in the range of from 100 to 180 mm, in particular about 160 mm.

In one embodiment, the coating device is a modified fluidizing bed, as illustrated in FIG. 1. The fluidized bed has a container 1 of nonconductive material having a bottom 2 and end walls 3. Optionally, a porous membrane 4 is positioned above the bottom 2 of the fluidized bed container 1 which, permits passage of a fluidizing gas, such as air, under pressure, and prevents the powder falling down. Alternatively, an electrostatic grid with a suitable arrangement of electrode wires can be used instead of the porous membrane.

Powder 5 in the container is fluidized by the passage of the fluidizing gas through an inlet 6 in the fluidizing container 1. Fluidizing gas may be supplied by a conventional air compressor connected to the inlet 6. Preferably, the inlet 6 in the container 1 is positioned between the bottom 1 and the porous membrane 4.

The coating device comprises at least two opposite-facing electrodes, wherein at least first electrode 7 is positioned above the porous material 8, and wherein at least second electrode 9 is positioned below the surface of the fluidized powder. Preferably, the electrodes are arranged to be essentially parallel to each other and to the porous material 8. The at least second electrode can be in a form of a plurality of electrode wires or an electrostatic grid.

In one embodiment, the porous material in the method of making the composition comprising is in the form of a sheet or a rolled sheet. A rolled sheet is an advantageous form for continuous operating mode of coating the porous material with a powder.

In one embodiment, the porous material in the method of making the composition is essentially flat, i.e. the thickness of the porous material throughout the length and the width of the material does not deviate from the average thickness of the porous material by more than +20%, preferably +10%. For example, with an average thickness of 1 mm the thickness of the material throughout its length and width remains in the range of from 0,8 to 1.2 mm, preferably in the range of from 0,9 to 1.1 mm. With an average thickness of 2 mm the thickness of the essentially flat material throughout its length and width remains in the range of from 1,6 to 2.4 mm, preferably in the range of from 1,8 to 2.2 mm.

In one embodiment, the porous material in the method of making the composition comprises at least 90% by weight biomaterial, preferably the porous material comprises at least 95% by weight biomaterial, more preferably the porous material comprises at least 96% by weight of biomaterial, more preferably the porous material comprises at least 97% by weight of biomaterial, more preferably the porous material comprises at least 98% by weight of biomaterial, most preferred the porous material comprises at least 99% by weight biomaterial. In one embodiment, the biomaterial is collagen.

In one embodiment the density of the porous material in the method of making the composition is in the range of from 0.01 to 1 g/cm³, preferably is in the range of from 0,02 to 0.05 g/cm³, more preferably in the range of from 0,02 to 0,04 g/cm³, most preferred in the range of from 0,022 to 0,03 g/cm³.

In one embodiment, the pores in the porous material in the method of making the composition have an average diameter in the range of from 15 to 70 μm, preferably in the range of from 25 to 65 μm.

In one embodiment the porous material in the method of making the composition has a density in the range of from 0.01 to 1 g/cm³, wherein the pores have an average diameter in the range of from 15 to 70 μm. In a preferred embodiment the porous material in the method of making the composition has a density in the range of from 0,02 to 0.05 g/cm³, wherein the pores have an average diameter in the range of from 25 to 65 μm. In a more preferred embodiment the porous material in the method of making the composition has a density in the range of from 0,02 to 0,04 g/cm³, wherein the pores have an average diameter in the range of from 25 to 65 μm.

In one embodiment the particles of the electrostatically chargeable powder in the method of making the composition comprising a porous material have an average size in the range of from 50 to 100 μm, preferably in the range of from 55 to 85 μm.

In one embodiment, the porous material in the method of making the composition is a biomaterial.

In one embodiment, the porous material in the method of making the composition is selected from the group comprising natural and/or synthetic polymers or mixtures thereof, in particular polysaccharides, glucosaminoglycanes, proteins or mixtures thereof.

In one embodiment, the porous material in the method of making the composition is selected from the group consisting of collagen, alginate, e.g. calcium alginate or a mixture thereof.

In one embodiment, the porous material in the method of making the composition is collagen.

In one embodiment, the porous material in the method of making the composition is alginate, in particular calcium alginate.

In one embodiment the porous material in the method of making the composition is a mixture of collagen and calcium alginate. In one embodiment, the porous material comprises collagen by weight in the range of from 80 to 98% and calcium alginate by weight in the range of from 2 to 20%. In a preferred embodiment, the porous material comprises collagen by weight in the range of from 85 to 95% and calcium alginate by weight in the range of from 5 to 15%. In particular, the porous material comprises collagen about 90% by weight and calcium alginate about 10% by weight.

In one embodiment, the collagen in the porous material in the method of making the composition comprising a porous material is animal derived native collagen with a triple helical structure.

In one embodiment the collagen in the porous material in the method of making the composition is selected from the group comprising type 1 collagen, type 3 collagen, type 5 collagen or a mixture thereof.

In one embodiment, the electrostatically chargeable powder in the method of making the composition comprising a porous material comprises a compound selected from the group consisting of a salt, a glucose polysaccharide, glucose, a modified glucose, an enzyme, a collagen, hyaluronic acid, a metal or a metal oxide.

In one embodiment, the electrostatically chargeable powder in the method of making the composition comprises a salt selected from the group comprising sodium bicarbonate (NaHCO₃), magnesium carbonate (MgCO₃), calcium carbonate (CaCO₃), sodium lactate, sodium citrate and sodium iodide (Nal), or a mixture thereof. In one embodiment, the electrostatically chargeable powder comprises a salt selected from the group comprising magnesium carbonate (MgCO₃), calcium carbonate (CaCO₃), sodium lactate, sodium citrate and sodium iodide (Nal), or a mixture thereof.

In one embodiment, the electrostatically chargeable powder in the method of making the composition comprises a salt, which is not sodium bicarbonate (NaHCO₃).

In one embodiment the salt in the method of making the composition comprising a porous material has a monovalent cation and a monovalent anion, for example, sodium bicarbonate (NaHCO₃). In one embodiment, the salt has a divalent cation and a monovalent anion, for example calcium carbonate (CaCO₃).

In one embodiment the salt in the method of making the composition comprising a porous material is sodium bicarbonate (NaHCO₃). Preferably, the salt comprises sodium bicarbonate (NaHCO₃) at least 95% by weight, more preferably at least 96% by weight, even more preferably at least 97% by weight, even more preferably at least 98% by weight, even more preferably at least 99% by weight, in particular at least 99,5% by weight with respect to the dry compound. The salt comprising sodium bicarbonate may contain minor amounts of sodium iodide (Nal) and/or magnesium carbonate (MgCO₃). In one embodiment, the salt comprising sodium bicarbonate contains up to 5% by weight sodium iodide (Nal) with respect to the dry compound. In one embodiment, the salt comprising sodium bicarbonate contains up to 5% by weight magnesium carbonate (MgCO₃) with respect to the dry compound. In one embodiment the salt consists of sodium bicarbonate (NaHCO₃).

In one embodiment sodium bicarbonate comprises less than 40% moisture by weight, preferably less 35% moisture by weight, more preferably less than 30% moisture by weight.

In one embodiment the electrostatically chargeable powder in the method of making the composition comprising a porous material comprises the glucose polysaccharide is selected from the group comprising cellulose and starch.

In one embodiment the electrostatically chargeable powder in the method of making the composition comprising a porous material comprises a modified glucose. In one embodiment the modified glucose is glucose with the radionuclide fluorine-18 (18F) in place of the hydroxyl group on the 2 carbon (FDG).

In one embodiment the electrostatically chargeable powder in the method of making the composition comprising a porous material comprises an enzyme.

In one embodiment the electrostatically chargeable powder in the method of making the composition comprising a porous material comprises collagen. In one embodiment the collagen in the porous material of the composition is selected from the group comprising type 1 collagen, type 3 collagen, type 5 collagen or a mixture thereof.

In one embodiment the electrostatically chargeable powder comprises hyaluronic acid.

In one embodiment the electrostatically chargeable powder in the method of making the composition comprising a porous material comprises a metal, such as is titanium.

In one embodiment the electrostatically chargeable powder in the method of making the composition comprising a porous material comprises a metal oxide, for example, titanium dioxide (TiO₂).

In one embodiment, the method according to any of the preceding embodiments further comprises a step of coating the composition of step d) with a layer of a polymer or wax. The examples of polymers include polyurethane and polyalkylene oxide polymers. In one embodiment the polymer is a polyalkylene oxide polymer, preferably a PEG comprising polymer, e.g. a multi-electrophilic polyalkylene oxide polymer, e.g. a multi-electrophilic PEG, such as pentaerythritolpoly(ethyleneglycol) ether tetrasuccinimidyl glutarate (COH 102). Suitable methods are described in EP 2939697 B1. For example, the polymer can be melted, and sprayed or printed onto the matrix of the biomaterial. Alternatively, it is also possible to sprinkle a dry form (e.g. a powder) of the polymer onto the matrix. If necessary, an increase of the temperature can be applied to achieve a permanent coating of the sponge. Alternatively, the polymer can be dissolved into inert organic solvents and brought onto the matrix of the biomaterial.

In a third aspect, the invention relates to a method of controlling bleeding and/or leakage of other body fluids in surgical procedures or for treatment of an injury selected from the group consisting of a wound, a hemorrhage, damaged tissue and/or bleeding tissue comprising administering a composition according to any of the preceding embodiments, in particular the composition coated with an additional layer of a polymer, such as polyalkylene oxide polymer, to a subject in the need thereof.

In a forth aspect, the invention relates to a method of treatment or prevention of wrinkles, skin irritation and other applications in the field of cosmetics and skin care comprising administering a composition according to any of the preceding embodiments to a subject in the need thereof.

In a fifth aspect, the invention relates to a composition comprising a porous material according to any of the preceding embodiments, in particular the composition coated with an additional layer of a polymer, such as polyalkylene oxide polymer, for use in controlling bleeding and/or leakage of other body fluids in surgical procedures or for treatment of an injury selected from the group consisting of a wound, a hemorrhage, damaged tissue and/or bleeding tissue.

In a sixth aspect, the invention relates to a use of a composition according to any of the preceding embodiments for treatment or prevention of wrinkles, skin irritation and other types of cosmetic skin treatment.

With the above context, the following consecutively numbered embodiments provide further specific aspects of the invention:
1. A composition comprising a porous material, wherein said porous material comprises a biomaterial and comprises a plurality of open and interconnected pores with pore surfaces,
   wherein said porous material has a density in the range of from 0.01 to 1 g/cm³, preferably in the range of from 0,02 to 0.05 g/cm³, in particular in the range of from of 0,02 to 0,04 g/cm³,
   wherein said pores have an average diameter in the range of from 15 to 70 μm, preferably in the range of from 25 to 65 μm, characterized in that said porous material is coated with an electrostatically chargeable powder comprising particles,
   wherein said particles have an average size in the range of from 50 to 100 μm, and
   wherein the total quantity of the coating is in the range of from 2 to 100 g/m², preferably in the range of from 3,5 to 9 g/m².
2. The composition comprising a porous material according to embodiment 1, wherein the average size of at least 70%, preferably of at least 80%, in particular of at least 90% of said particles exceeds the average diameter of said pores.
3. The composition comprising a porous material according to embodiment 1 or 2, wherein said porous material is a biomaterial.
4. The composition comprising a porous material according to any of embodiments 1 to 3, wherein said porous material is selected from the group comprising natural and/or synthetic polymers or mixtures thereof, in particular polysaccharides, glucosaminoglycanes, proteins and/or synthetic polymers or mixtures thereof.
5. The composition comprising a porous material according to any of embodiments 1 to 4, wherein said porous material is selected from the group consisting of collagen, alginate or a mixture thereof.
6. The composition comprising a porous material according to embodiment 5, wherein said biomaterial is collagen.
7. The composition comprising a porous material according to embodiment 6, wherein said porous material is animal derived native collagen with a triple helical structure.
8. The composition comprising a porous material according to any of embodiments 1 to 7, wherein said composition is in the form of a sheet or a 3D form.
9. The composition comprising a porous material according to any of embodiments 1 to 8, wherein the coating adjusts the pH at the surface of the composition and the surface of the pores to a range of from 3,0 to 9,0, preferably in the range of from 6,0 to 8,0.
10. The composition comprising a porous material according to embodiment 9, wherein the pH at the surface is measured by a surface pH electrode, in particular a pH electrode with a flat membrane and polymer electrolyte.
11. The composition comprising a porous material according to any of embodiments 1 to 10, wherein said powder comprises a compound selected from the group consisting of a salt, a glucose-based polysaccharide, glucose, a modified glucose, an enzyme, a collagen, hyaluronic acid, a metal or a metal oxide.
12. The composition comprising a porous material according to any of embodiments 1 to 11, wherein said powder comprises a compound that is sodium bicarbonate (NaHCO₃).
13. The composition comprising a porous material according to embodiment 12, wherein said powder contains at least 95% by weight of sodium bicarbonate NaHCO₃.
14. The composition comprising a porous material according to any of embodiments 1 to 13, which is coated with a layer of a polymer or a wax.
15. A method of making the composition comprising a porous material as defined in any of embodiments 1 to 13, comprising the steps of:
   a) Providing a porous material as defined in embodiment 1;
   b) Proving an electrostatically chargeable powder comprising particles, wherein said particles have an average size in the range of from 50 to 100 μm;
   c) Positioning said porous material and said powder between opposite-facing electrodes in a coating device, wherein said coating device is able to generate an electric field through the porous medium, and wherein said device has an area for storing said powder;
   d) Electrostatic depositing of said powder on the surface of said porous material by means of subjecting said powder and said porous material to an electric field produced by the opposing electrodes, wherein said electric field moves said particles towards said porous material.
16. The method of making the composition comprising a porous material according to embodiment 15, wherein said powder in step d) is in a fluidized state by means of providing a stream of a fluidizing gas into said area for storing said powder in said device.
17. The method of making the composition comprising a porous material according to embodiment 15 or 16, wherein the voltage applied to said opposing-facing electrodes is in the range of from 20 to 250 kV, preferably in the range of from 30 to 60 kV, more preferably in the range of from 45 to 55 kV, in particular about 50 kV.
18. The method of making the composition comprising a porous material according to any of embodiments 15 to 17, wherein the opposing-facing electrodes in said coating device are arranged in the way that at least first electrode, which is anode, is positioned in a proximity to said porous material, and at least second electrode, which is cathode, is positioned in a proximity to said area for storing powder in said device.
19. The method of making the composition comprising a porous material according to embodiment 18, wherein the at least second electrode, which is cathode, is an electrostatic grid.
20. Method of making the composition comprising a porous material according to embodiment 18 or 19, wherein said particles are charged at the cathode and are electrostatically attracted by the anode.
21. Method of making the composition comprising a porous material according to any of embodiments 15 to 20, wherein said coating device is connected to a voltage generator.
22. Method of making the composition comprising a porous material according to any of embodiments 15 to 21, wherein said coating device comprises a means for moving said porous material horizontally across said powder to at least partly displace a portion of said powder across said porous material.
23. Method of making the composition comprising a porous material according to embodiment 22, wherein said means for moving said porous material is at least one roll.
24. The method of making the composition comprising a porous material according to any of embodiments 16 to 23, wherein the volumetric flow rate of said fluidizing gas is in the range of from 40 to 120 l/min, preferably in the range of from 60 to 100 l/min, in particular about 80 l/min.
25. The method of making the composition comprising a porous material according to any of embodiments 15 to 24, wherein the density of said powder is in the range of from 2,0 to 2,5 g/cm³, preferably in the range of from 2,1 to 2,3 g/cm³, in particular about 2,2 g/cm³.
26. The method of making the composition comprising a porous material according to any of embodiments 15 to 25, wherein the distance between said area for storing said powder and the porous material in the coating device is in the range of from 80 to 200 mm, preferably in the range of from 100 to 180 mm, in particular about 160 mm.
27. The method of making the composition comprising a porous material according to any of embodiments 15 to 26, wherein said porous material is in the form of a sheet or a rolled sheet.
28. The method of making the composition comprising a porous material according to any of embodiments 15 to 27, wherein said porous material is a biomaterial.
29. The method of making the composition comprising a porous material according to any of embodiments 15 to 28, wherein said porous material is selected from the group comprising natural and/or synthetic polymers or mixtures thereof, in particular polysaccharides, glucosaminoglycanes, proteins or mixtures thereof.
30. The method of making the composition comprising a porous material according to any of embodiments 15 to 29, wherein said porous material is selected from the group consisting of collagen, alginate or a mixture thereof.
31. The method of making the composition comprising a porous material according to embodiment 30, wherein said porous material is collagen.
32. The method of making the composition comprising a porous material according to embodiment 31, wherein said porous material is animal derived native collagen with a triple helical structure.
33. The method of making the composition comprising a porous material according to any of embodiments 15 to 32, wherein said powder comprises a compound selected from the group consisting of a salt, a glucose-based polysaccharide, glucose, a modified glucose, an enzyme, a collagen, a metal or a metal oxide.
34. The method of making the composition comprising a porous material according to any of embodiments 15 to 33, wherein said powder comprises a compound that is sodium bicarbonate (NaHCO₃).
35. The method of making the composition comprising a porous material according to any of embodiments 15 to 34, wherein said powder contains at least 95% by weight of sodium bicarbonate (NaHCO₃).
36. The method of making the composition comprising a porous material according to any of embodiments 15 to 35, further comprising a step of coating the composition of step d) with a layer of a polymer or a wax.
37. A method of controlling bleeding and/or leakage of other body fluids in surgical procedures or for treatment of an injury selected from the group consisting of a wound, a hemorrhage, damaged tissue and/or bleeding tissue comprising administering a composition comprising a porous material as defined in any of embodiments 1 to 14 to a subject in the need thereof.
38. A method of treatment or prevention of wrinkles, skin irritation and other applications in the field of cosmetics and skin care comprising administering the composition comprising a porous material as defined in any of embodiments 1 to 14 to a subject in the need thereof.
39. The composition comprising a porous material as defined in any of embodiments 1 to 14 for use in controlling bleeding and/or leakage of other body fluids in surgical procedures or for treatment of an injury selected from the group consisting of a wound, a hemorrhage, damaged tissue and/or bleeding tissue.
40. The composition comprising a porous material as defined in any of embodiments 1 to 14 for use in treatment or prevention of wrinkles, skin irritation and other applications in the field of cosmetics and skin care.
41. Use of the composition comprising a porous material as defined in any of embodiments 1 to 14 for treatment or prevention of wrinkles, skin irritation and other applications in the field of cosmetics and skin care.
42. A composition comprising a porous material, wherein said porous material comprises collagen and comprises a plurality of open and interconnected pores with pore surfaces,
   wherein said porous material has a density in the range of from 0.01 to 1 g/cm³, preferably in the range of from 0,02 to 0.05 g/cm³, in particular in the range of from of 0,02 to 0,04 g/cm³,
   wherein said pores have an average diameter in the range of from 15 to 70 μm, preferably in the range of from 25 to 65 μm, characterized in that said porous material is coated with an electrostatically chargeable powder comprising particles,
   wherein said powder contains at least 95% by weight of sodium bicarbonate (NaHCO₃),
   wherein said particles have an average size in the range of from 50 to 100 μm, wherein the total quantity of the coating is in the range of from 2 to 100 g/m² of the outer surface of said porous material, preferably in the range of from 3,5 to 9 g/m² of the outer surface of said porous material, and
   wherein the coating adjusts the pH at the surface of the composition to a range of from 3,0 to 9,0, preferably in the range of from 6,0 to 8,0.
43. The composition comprising a porous material according to embodiment 42, wherein the average size of at least 70% of said particles, preferably of at least 80% said particles, in particular of at least 90% of said particles exceeds the average diameter of said pores.
44. The composition comprising a porous material according to embodiment 42 or 43, wherein the pH at the surface is measured by a surface pH electrode, in particular a pH electrode with a flat membrane and polymer electrolyte.
45. The composition comprising a porous material according to any of embodiments 42 to 44, wherein said porous material is animal derived native collagen with a triple helical structure.
46. The composition comprising a porous material according to any of embodiments 42 to 45, wherein said composition is in the form of a sheet or a 3D form.
47. The composition comprising a porous material according to any of embodiments 42 to 46, which is additionally coated with a layer of a polyalkylene oxide polymer for use in controlling bleeding and/or leakage of other body fluids in surgical procedures.
48. A method of making the composition comprising a porous material as defined in any of embodiments 42 to 46, comprising the steps of:
   a) Providing a porous material as defined in embodiment 1;
   b) Proving an electrostatically chargeable powder comprising particles, wherein said powder contains at least 95% by weight of sodium bicarbonate (NaHCO₃) and wherein said particles have an average size in the range of from 50 to 100 μm;
   c) Positioning said porous material and said powder between opposite-facing electrodes in a coating device, wherein said coating device is able to generate an electric field through the porous medium, and wherein said device has an area for storing said powder;
   d) Electrostatic depositing of said powder on the surface of said porous material by means of subjecting said powder and said porous material to an electric field produced by the opposing electrodes, wherein said electric field moves said particles towards said porous material.
49. The method of making the composition comprising a porous material according to embodiment 48, wherein said powder in step d) is in a fluidized state by means of providing a stream of a fluidizing gas into said area for storing said powder in said device.
50. The method of making the composition comprising a porous material according to embodiment 48 or 49, wherein the voltage applied to said opposing-facing electrodes is in the range of from 20 to 250 kV, preferably in the range of from 30 to 60 kV, more preferably in the range of from 45 to 55 kV, in particular about 50 kV.
51. The method of making the composition comprising a porous material according to any of embodiments 48 to 50, wherein the opposing-facing electrodes in said coating device are arranged in the way that at least first electrode, which is anode, is positioned in a proximity to said porous material, and at least second electrode, which is cathode, is positioned in a proximity to said area for storing powder in said device.
52. The method of making the composition comprising a porous material according to embodiment 51, wherein the at least second electrode, which is cathode, is an electrostatic grid.
53. The method of making the composition comprising a porous material according to embodiment 51 or 52, wherein said particles are charged at the cathode and are electrostatically attracted by the anode.
54. The method of making the composition comprising a porous material according to any of embodiments 48 to 53, wherein said coating device is connected to a voltage generator.
55. The method of making the composition comprising a porous material according to any of embodiments 48 to 54, wherein said coating device comprises a means for moving said porous material horizontally across said powder to at least partly displace a portion of said powder across said porous material.
56. The method of making the composition comprising a porous material according to embodiment 55, wherein said means for moving said porous material is at least one roll.
57. The method of making the composition comprising a porous material according to any of embodiments 49 to 56, wherein the volumetric flow rate of said fluidizing gas is in the range of from 40 to 120 l/min, preferably in the range of from 60 to 100 l/min, in particular about 80 l/min.
58. The method of making the composition comprising a porous material according to any of embodiments 48 to 57, wherein the density of said powder is in the range of from 2,0 to 2,5 g/cm³, preferably in the range of from 2,1 to 2,3 g/cm³, in particular about 2,2 g/cm³.
59. The method of making the composition comprising a porous material according to any of embodiments 48 to 58, wherein the distance between said area for storing said powder and the porous material in the coating device is in the range of from 80 to 200 mm, preferably in the range of from 100 to 180 mm, in particular about 160 mm.
60. The method of making the composition comprising a porous material according to any of embodiments 48 to 59, wherein said porous material is in the form of a sheet or a rolled sheet.
61. The method of making the composition comprising a porous material according to any of embodiments 48 to 60, wherein said porous material is animal derived native collagen with a triple helical structure.
62. The method of making the composition comprising a porous material according to any of embodiments 48 to 61, further comprising a step of coating the composition of step d) with a layer of a polyalkylene oxide polymer.
63. A method of controlling bleeding and/or leakage of other body fluids in surgical procedures comprising administering the composition comprising a porous material as defined in embodiment 47 to a subject in the need thereof.

### Definitions

Listed below are definitions of various terms used to describe this invention. These definitions apply to the terms as they are used throughout this specification and claims unless otherwise limited in specific instances either individually or as part of a larger group. Unless defined otherwise all technical and scientific terms used herein generally have the same meaning as commonly understood by one of ordinary skill in the art to which this invention belongs.

As used herein, the articles “a” and “an” refer to one or to more than one (i.e. to at least one) of the grammatical object of the article. By way of example, “an element” means at least one element, i.e. an element or more than one element.

As used herein, the term “porous material” refers to a material comprising pores, i.e. cavities, channels or interstices, wherein the depth of the pores exceeds their average diameter.

Unless specifically defined, the term “thickness” related to a porous material refers to an average thickness of the porous material.

As used herein, the term “electrostatically chargeable powder” means a powder that is ionically chargeable by electrostatic induction means.

As used herein, the term “essentially flat” refers to the material with the thickness throughout the length and the width not deviating from the average thickness by more than +20%, preferably +10%.

As used herein, the term “sheet” refers to an essentially flat material, wherein the thickness of the material is in the range of from 1 to 8 mm.

As used herein, the terms “rolled material” and “rolled sheet” are used interchangeably and refer to the sheet of porous material wound cylindrically about a center axis of a roll with the possibility of removal of the material from the center or inner periphery of the roll.

As used herein, the term “3D form” refers to any form of the porous material which is not a sheet or a rolled sheet.

As used herein, the term “dressing” refers to the composition comprising a porous material coated with a powder, which is further coated with an additional layer of a polymer, for example on top of the powder coating, wherein said composition is in a form of a sheet.

As used herein, the term “coating” refers to a thin deposit of a material that substantially covers the surface of a substrate.

As used herein, the term “wax” refers to hydrogenated forms of naturally occurring vegetable oils and/or animal fats.

As used herein in, the term “anode” refers to the negative electrode from which electrons flow during the discharging phase in the battery. The anode is the electrode that undergoes chemical oxidation during the discharging phase and chemical reduction in the charging phase.

As used herein, the term “cathode” refers to the positive electrode into which electrons flow during the discharging phase in the battery. The cathode is the electrode that undergoes chemical reduction during the discharging phase and chemical oxidation during the charging phase.

As used herein, the term “salt” refers to ionic a chemical compound consisting of an ionic assembly of a positively charged cation and a negatively charged anion. Non-limiting examples of salts include sodium bicarbonate (NaHCO₃), magnesium carbonate (MgCO₃), calcium carbonate (CaCO₃), sodium lactate, sodium citrate and sodium iodide (NaI)

As used herein, the term “biomaterial” refers to a natural or synthetic biocompatible material which is suitable for using in a medical device, intended to interact with biological systems. Non-limiting examples include collagen, gelatine, alginate and polysaccharides such as glycosaminoglycans.

As used herein, the term “polysaccharide” refers to polymers comprising a backbone comprised mainly of (at least 90%) monosaccharide repeating units and/or derivatized monosaccharide repeating units.

As used herein, the term “glucose polysaccharide” refers to polymers comprising a backbone comprised mainly of (at least 90%) glucose repeating units and/or derivatized glucose repeating units. Non-limiting examples include starches, modified starches, cellulose, modified cellulose.

As used herein, the term “modified glucose” refers to glucose, in which at least one OH-group is replaced with a group which is not OH-group, or a hydrogen atom in at least one OH-group is replaced with an atom which is not hydrogen.

As used herein, the term “protein” or “polypeptide” refers to a polymer of two or more of the natural amino acids or non-natural amino acids.

As used herein the term “enzyme” refers to any protein that catalyses a chemical reaction. An enzyme typically is classified according to the type of catalytic function it carries out, e.g. hydrolysis of bonds (“hydrolases”), isomerization “isomerases”, etc.

As used herein the term “glycosaminoglycan” refers to a group of acid polysaccharides, each having a repeating unit of disaccharide consisting of an amino sugar and uronic acid or galactose.

As used herein, the term “alginate” refers to the anion of alginic acid. Therefore, the terms “alginate” and “alginate salt” are used interchangeable in the context of the present invention. The alginate salt can be, for example, calcium alginate. Alginate is a linear polymer formed by anions of β-D-mannuronic acid (M, β-D-mannuronate) and of α-L-guluronic acid (G, α-L-guluronate) bound by means of 1-4 glycosidic bonds.

As used herein, the term “collagen” refers to the extracellular family of fibrous proteins that are characterised by their stiff, triple-stranded helical structure. Three collagen polypeptide chains (“α-chains”) are wound around each other to form this helical molecule.

As used herein, the terms “parallel” and “essentially parallel” have a tolerance of +10°.

As used herein the term “subject” refers to a human or a non-human mammal. Preferably the subject is human.

### DESCRIPTION OF FIGURES

FIG. 1 shows a coating device which is a modified fluidizing bed.

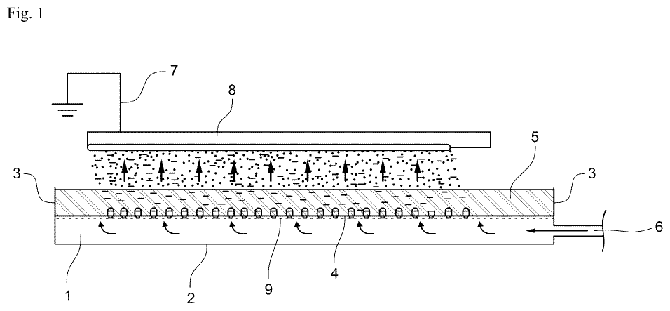

### EXAMPLES

The invention is now described with the reference to the following non-limiting Examples.

### Example 1

0,3-0,4 g NaHCO₃ with the particle size ranging between 10 and 200 μm was sieved with the sieving diameter range between 50 and 100 μm. The sieved powder was filled into the basin of the fluidized bed with a layer of up to 30 mm. The collagen sheet with the thickness of 2 mm, the density of 0,02-0,04 g/cm³ and with the average diameter of pores of 15-70 μm was clamped over the coating basin at a distance of the powder level of 160 mm. The clean, pressurized air is applied at a volumetric rate of 79 l/min to fluidize the powder. The voltage of −50 kV is applied in the fluid bed for 3 seconds to coat the powder onto the collagen.

### Example 2

A comparison of properties of the collagen sheet coated with NaHCO₃ obtained by a method according to Example 1 to uncoated collagen material:

TABLE 1

| | | |
|---|---|---|
| | Collagen coated | Uncoated |
| Parameter | with NaHCO₃ | collagen |

| | | |
|---|---|---|
| Residual moisture | 11.27% | 12.31% |
| Water absorption | 28.39 g per g of | 27.70 g per g of |
| | collagen | collagen |

| | | | | |
|---|---|---|---|---|
| Tensile strength | 433.79 | cN | 378 | cN |
| Thickness (average | 2.06 | mm | 2.10 | mm |
| of 6 samples) | | | | |

As can be seen in table 1, the overall properties of the coated and uncoated material are essentially the same. The deviations from each other are also seen within different batches of uncoated collagen only.

1. A composition comprising a porous material, wherein said porous material comprises a biomaterial and comprises a plurality of open and interconnected pores with pore surfaces,
wherein said porous material has a density in the range of from 0.01 to 1 g/cm³, preferably in the range of from 0,02 to 0.05 g/cm³, in particular in the range of from of 0,02 to 0,04 g/cm³,
wherein said pores have an average diameter in the range of from 15 to 70 μm, preferably in the range of from 25 to 65 μm,
characterized in that said porous material is coated with an electrostatically chargeable powder comprising particles,
wherein said particles have an average size in the range of from 50 to 100 μm, and,
wherein the total quantity of the coating is in the range of from 2 to 100 g/m², preferably in the range of from 3,5 to 9 g/m².

2. The composition comprising a porous material according to claim 1, wherein the average size of at least 70%, preferably of at least 80%, in particular of at least 90% of said particles exceeds the average diameter of said pores.

3. The composition comprising a porous material according to claim 1, wherein said porous material is selected from the group comprising natural and/or synthetic polymers or mixtures thereof, in particular polysaccharides, glucosaminoglycanes, proteins and/or synthetic polymers or mixtures thereof.

4. The composition comprising a porous material according to claim 1, wherein said porous material is selected from the group consisting of collagen, alginate or a mixture thereof.

5. The composition comprising a porous material according to claim 1, wherein the coating adjusts the pH at the surface of the composition and the surface of the pores to a range of from 3,0 to 9,0, preferably in the range of from 6,0 to 8,0.

6. The composition comprising a porous material according to claim 1, wherein said powder comprises a compound selected from the group consisting of a salt, a glucose-based polysaccharide, glucose, a modified glucose, an enzyme, a collagen, hyaluronic acid, a metal or a metal oxide.

7. The composition comprising a porous material according to claim 1, wherein said powder comprises a compound that is sodium bicarbonate (NaHCO₃).

8. The composition comprising a porous material according to claim 1, which is coated with a layer of a polymer or a wax.

9. A method of making the composition comprising a porous material as defined in claim 1, comprising the steps of:
a) providing a porous material as defined in claim 1;
b) proving an electrostatically chargeable powder comprising particles, wherein said particles have an average size in the range of from 50 to 100 μm;
c) positioning said porous material and said powder between opposite-facing electrodes in a coating device,
wherein said coating device is able to generate an electric field through the porous medium, and
wherein said device has an area for storing said powder;
d) electrostatic depositing of said powder on the surface of said porous material by means of subjecting said powder and said porous material to an electric field produced by the opposing electrodes,
wherein said electric field moves said particles towards said porous material.

10. The method of making the composition comprising a porous material according to claim 9, wherein said powder in step d) is in a fluidized state by means of providing a stream of a fluidizing gas into said area for storing said powder in said device.

11. The method of making the composition comprising a porous material according to claim 9, wherein the voltage applied to said opposing-facing electrodes is in the range of from 20 to 250 kV, preferably in the range of from 30 to 60 kV, more preferably in the range of from 45 to 55 kV, in particular about 50 kV.

12. The method of making the composition comprising a porous material according to claim 9, wherein the opposing-facing electrodes in said coating device are arranged in the way that at least first electrode, which is anode, is positioned in a proximity to said porous material, and at least second electrode, which is cathode, is positioned in a proximity to said area for storing powder in said device.

13. The method of making the composition comprising a porous material according to claim 9, wherein the density of said powder is in the range of from 2,0 to 2,5 g/cm³, preferably in the range of from 2,1 to 2,3 g/cm³, in particular about 2,2 g/cm³.

14. The method of making the composition comprising a porous material according to claim 9, further comprising a step of coating the composition of step d) with a layer of a polymer or a wax.

15. A method of controlling bleeding and/or leakage of other body fluids in surgical procedures or for treatment of an injury selected from the group consisting of a wound, a hemorrhage, damaged tissue and/or bleeding tissue comprising administering a composition comprising a porous material as defined in claim 1 to a subject in the need thereof.
